(19) United States
(12) Patent Application Publication
(10) Pub. No.: US 2026/0325463 A1
(43) Pub. Date: Oct. 22, 2026

(54) TREATMENT OF OSTEOARTHRITIS USING COMT INHIBITORS

(71) Applicants: UNIVERSITE DE CAEN NORMANDIE, Caen (FR); INSTITUT NATIONAL DE LA SANTÉ ET DE LA RECHERCHE MÉDICALE, Paris (FR); CENTRE HOSPITALIER UNIVERSITAIRE DE CAEN NORMANDIE, Caen (FR); UNIVERSITE PARIS CITE, Paris (FR)

(72) Inventors: Catherine BAUGE, Caen Cedex 5 (FR); Juliette AURY-LANDAS, Caen Cedex 5 (FR); Sybille BROCHARD, Caen Cedex 5 (FR); Karim BOUMEDIENNE, Caen Cedex 5 (FR); Anthony GROSO, Orléans (FR); Quitteric BRIET-ROCHOUX, Caen (FR); Martine COHEN-SOLAL, Paris (FR); Éric HAY, Paris (FR); Zohra BOUCHEMLA, Paris (FR); Alison VANLAEYS, Colombelles (FR)

(21) Appl. No.: 19/122,039

(22) PCT Filed: Oct. 25, 2023
(86) PCT No.: PCT/EP2023/079822

§ 371 Date: Apr. 17, 2025

(30) Foreign Application Priority Data
Oct. 26, 2022 (EP) 22306622.6

(51) Int. Cl. A61K 31/12; A61K 9/00; A61K 31/277; A61K 31/4439; A61K 31/56; A61K 31/7008; A61K 31/7105; A61K 31/737; A61K 35/19; A61K 38/00; A61K 45/06; A61P 19/02; C07K 16/00

(52) U.S. Cl. CPC A61K 31/12; A61K 9/0019; A61K 31/277; A61K 31/4439; A61K 31/56; A61K 31/7008; A61K 31/7105; A61K 31/737; A61K 35/19; A61K 38/00; A61K 45/06; A61P 19/02; C07K 16/00

(57) ABSTRACT

The invention relates to a catechol O-methyltransferase (COMT) inhibitor for use in the treatment of osteoarthritis, the inhibitor being intended to slow down the progression of osteoarthritis and reduce osteoarthritis pain, as well as to a pharmaceutical composition comprising a COMT inhibitor for the treatment of osteoarthritis.

### TECHNICAL FIELD

The present invention is directed toward providing a new strategy for treating osteoarthritis.

### TECHNOLOGICAL BACKGROUND

Osteoarthritis or chronic degenerative arthropathy is a chronic joint disease characterized by structural deterioration of articular cartilage. Apart from the spine, the joints most affected are the knee, hand and hip.

The symptoms of this pathology may range according to the joint concerned, but are generally characterized by persistent pain associated with functional discomfort, i.e. a limitation in the mobility of the joint concerned.

Articular cartilage is a connective tissue made up of a single cell type, chondrocytes, which secrete an extracellular matrix and ensure its renewal. This matrix is essentially made up of water, proteoglycans and collagen.

Chondrocytes maintain a balance between degradation and restoration of the extracellular matrix. These mechanisms are complex and notably governed, for degradation, by pro-inflammatory cytokines such as TNF-α and IL-1β, and for restoration, by modulating cytokines and growth factors, notably IGF-1, TGF-β.

Arthritic destruction of cartilage results from a breakdown in this balance between degradation and restoration of the extracellular matrix. Several factors may contribute toward this breakdown in matrix homeostasis, notably mechanical factors, for example linked to cartilage hyperpressure or repeated microtrauma, or metabolic, genetic, hormonal factors, or aging. The initiation of the osteoarthritic process is still poorly understood.

At present, there is no effective, long-lasting treatment for osteoarthritis and its progression. The treatments used are purely symptomatic, with immediate or delayed action on the pain experienced, usually involving analgesics, anti-inflammatories and intra-articular infiltrations of hyaluronic acid or corticoids.

The development of new analgesics to relieve osteoarthritis pain in patients who are intolerant or refractory to standard analgesic treatments is complex. In particular, anti-NGF (Nerve Growth Factor) antibodies, which target a neurotrophin involved in pain signal transduction, have been the subject of numerous clinical studies. These anti-NGF agents have been shown to have a very clear pain-relieving effect, which is sometimes, unexpectedly and as yet unexplained, associated with an acceleration in the progression of osteoarthritis. There is no direct correlation between reduced pain and reduced progression of joint destruction.

The only effective curative treatment for mobility and pain is the insertion of a surgical prosthesis. Surgery is usually limited to hip, knee or shoulder joints, and very rarely to small joints such as the hands or feet. While hip prosthesis is the surgical treatment of reference, as it gives very good results, prosthetic replacement of other joints is unsatisfactory in terms of mobility and pain. Furthermore, the prostheses used have a limited lifespan of between 15 and 20 years, and have to be replaced if they are fitted in young patients.

At present, osteoarthritis is estimated to concern about nine to ten million people in France. Given the aging of the population and also the increasing prevalence of obesity in developed countries, a very sharp rise in the number of osteoarthritis patients is expected in the coming years. There is a need to find new therapeutic strategies that are capable of slowing down cartilage destruction in osteoarthritis patients, while at the same time reducing joint pain.

### SUMMARY

The object of the present invention is to provide novel compounds that can be used in the treatment of osteoarthritis.

The inventors have shown, surprisingly, that the COMT enzyme is expressed by joint chondrocytes, and that, even more surprisingly, COMT inhibitors administered to the joints are capable of slowing down or inhibiting the progression of cartilage destruction, and of reducing the pain associated with osteoarthritis via a local and direct action in the joints, through a mechanism that does not involve the central or peripheral nervous system.

Thus, according to a first subject, the invention relates to a catechol O-methyltransferase (COMT) inhibitor for use in the treatment of osteoarthritis.

Advantageously, the inhibitor is intended to slow down the progression of osteoarthritis and reduce the pain induced by osteoarthritis.

The inhibitor may be chosen from a compound derived from nitrocatechol, an antibody or an interfering RNA.

Advantageously, the inhibitor is a nitrocatechol derivative comprising a catechol part bearing a nitro group in the ortho or para position relative to one of the hydroxyl groups of the catechol part.

The nitrocatechol derivative is preferably chosen from the group consisting of tolcapone, entacapone, opicapone, nitecapone, nebicapone and mixtures thereof.

The inhibitor is preferably intended for intra-articular, oral or subcutaneous administration.

Preferably, the inhibitor is intended for intra-articular administration.

The inhibitor may be administered in combination with one or more active agents chosen from the group consisting of analgesics, non-steroidal anti-inflammatory drugs, steroidal anti-inflammatory drugs, and drugs for osteoarthritis, typically symptomatic slow-acting drugs for osteoarthritis or chondroprotectors.

The invention also relates to a pharmaceutical composition comprising one or more COMT inhibitors as defined in the first subject of the invention, and a pharmaceutically acceptable excipient, for use in the treatment of osteoarthritis.

Preferably, the pharmaceutical composition is intended for intra-articular, oral or subcutaneous administration.

The pharmaceutical composition may also comprise one or more other active substances chosen from the group consisting of analgesics, non-steroidal anti-inflammatory drugs, steroidal anti-inflammatory drugs and drugs for osteoarthritis.

Preferably, the pharmaceutical composition is intended for intra-articular administration.

The pharmaceutical composition may comprise the inhibitor and anti-arthritic agents such as platelet-rich plasma (PRP), chondroitin and glucosamine.

The pharmaceutical composition may be formulated in the form of a sustained-release composition.

The pharmaceutical composition may comprise the inhibitor dispersed in a polymeric fraction comprising a biodegradable and/or biocompatible polymer preferably chosen from the group consisting of hyaluronic acid, collagen, chitosan, polylactic acid (PLA), glycolic acid, copolymers of lactic acid and glycolic acid (PLGA), polyoxalates, polycaprolactone, polyesters, and mixtures thereof.

### BRIEF DESCRIPTION OF THE DRAWINGS

FIG. 1: Study of the messenger RNA and proteins in human articular chondrocytes and synoviocytes. The results show that COMT is expressed (both mRNA and protein) in cells present in the joint (human samples).

FIG. 2: Histological sections of arthritic mice and normal mice (DMM model). The results show that COMT expression is increased in the joints of osteoarthritic mice relative to normal mice (DMM model).

FIG. 3: A. Study of expression of the mRNA encoding MMP13 in chondrocytes. B. Study of expression of mRNA encoding IL-6 in chondrocytes. The results show that pharmacological inhibition of COMT by tolcapone reduces the expression of genes involved in the osteoarthritis process (inflammation, catabolism) in human articular chondrocytes.

FIG. 4A: Scheme illustrating the protocol comprising induction of osteoarthritis (Day 0) in a murine model of osteoarthritis (IOAM mice), repeated injections of the pharmacological COMT inhibitor (tolcapone on Days 7, 14, 21), monitoring of pain by the Von Frey technique on Days 12, 29, 37, 49 and 55 and by a posture test on Day 49, and killing on Day 56, for histological study of cartilage degradation.

FIG. 4B: Study of pain evaluated by the Von Frey technique as a function of time (day 0=osteoarthritis induction). The results show that intra-articular injections of a pharmacological COMT inhibitor (tolcapone) reduce pain (in this case, the threshold of paw withdrawal following mechanical stimulation) in a mouse model of osteoarthritis (IOAM).

FIG. 5: Histological sections of mouse knees in a murine model of osteoarthritis (IOAM mice) with and without injection of tolcapone. The results show that intra-articular injections of a pharmacological COMT inhibitor (tolcapone) reduce the progression of osteoarthritis in a mouse model of osteoarthritis (IOAM).

FIG. 6: Scheme illustrating static weight bearing 7 weeks after induction of osteoarthritis (IOAM). The results show that intra-articular injections of a pharmacological COMT inhibitor (tolcapone) improve the posture of osteoarthritic mice. Without tolcapone, the mice put less pressure on the arthritic paw than on the normal paw. After intra-articular injection of tolcapone into the osteoarthritic paw, the animals pressed similarly on both hind legs, confirming the pain reduction.

FIG. 7: Scheme illustrating the quantification of the degree of osteoarthritis according to the OARSI score in a mouse model of osteoarthritis (IOAM mice) with and without injection of tolcapone. The higher the score, the greater the degree of osteoarthritis.

FIG. 8: Scheme illustrating pain evaluated by the Von Frey technique 7 weeks after injection. The results show that intra-articular injections of a pharmacological COMT inhibitor (tolcapone) reduce pain in a mouse model of osteoarthritis (IOAM) at least as much as or even more than hyaluronic acid injections.

FIG. 9: Study of expression of the mRNA encoding IL-6 in synoviocytes. The results show that pharmacological inhibition of COMT by tolcapone reduces the expression of genes involved in the osteoarthritic process (inflammation) in human articular synoviocytes.

FIG. 10: Study of expression of the mRNA encoding COMT and MMP13 in human articular chondrocytes transfected with control siRNA (SiCtrl) or siRNAs directed against COMT (siCOMT) in the presence or absence of IL-1β. The results show that inhibition of COMT expression by siRNA reduces the expression of genes involved in the osteoarthritic process (catabolism) in human articular chondrocytes.

### DETAILED DESCRIPTION

Catechol O-methyltransferase (COMT) is the enzyme responsible for the O-methylation of endogenous neurotransmitters, xenobiotics and hormones incorporating catechol-based structures. Catecholamines are a family of molecules which act as hormones or neurotransmitters. The main catecholamines present in humans are adrenalin (or epinephrine), noradrenalin (or norepinephrine) and dopamine. They are produced by the adrenal glands and post-ganglionic neurons of the parasympathetic nervous system, by an enzyme called tyrosine hydroxylase (TH). Catecholamines are degraded by COMT, which catalyzes the transfer of a methyl group from S-adenosyl-L-methionine (SAM) to a hydroxyl group on a catechol nucleus. COMT exists in two forms: a long membrane-bound form which enables dopaminergic and noradrenergic synaptic neurotransmission to be stopped, and a short soluble form responsible for eliminating exogenous catechols.

COMT is a biological drug target for the treatment of various disorders of the central and peripheral nervous systems, notably Parkinson's disease, depression, schizophrenia and other diseases linked to dopamine deficiency.

In particular, drugs in the catechol-O-methyltransferase inhibitor (I-COMT) therapeutic class are indicated in the treatment of Parkinson's disease, as adjuvants for prolonging the duration of action of drugs containing dopamine precursors such as L-DOPA. Thus, I-COMTs used in Parkinson's disease are systematically prescribed in combination with L-DOPA, and have no symptomatic effect in monotherapy.

The inventors have shown that COMT inhibitors may have a therapeutic effect in the context of a completely different class of pathologies, namely osteoarthritis.

A role for COMT in pain, particularly osteoarthritis-related pain, has been mentioned by some authors, based on genetic association studies (see, for example, Meurs et al., Arthritis & Rheumatism. 60 628-629. 2009; Schutte et al., Research in Gerontological Nursing. Vol. 13, No. 4. 2020; Martire et al., Scandinavian Journal of Pain. 10 6-12. 2016; Govil et al., Eur J Pain. 24 398-412. 2020) but was nevertheless contested (see for example Olesen et al., Pain Pract. 18 587-596. 2018; Neogi et al., Ann Rheum Dis. 73 315-317. 2014). However, it was assumed that the mechanism of COMT inhibitors on pain was related to an effect on pain perception via the nervous system. In other words, COMT inhibitors were suggested as simple analgesics. A direct role for COMT in articulation had never been suggested. Prior to the inventors' work, the COMT enzyme had never been investigated as a target for the treatment of osteoarthritis, and in particular COMT expression within the joint had never been investigated.

The inventors have now demonstrated, surprisingly, that COMT is expressed in articular chondrocytes, and, even more surprisingly, that local and direct inhibition of COMT in the joints enables the progression of cartilage destruction to be reduced, slowed or inhibited simultaneously with a reduction in pain.

The inventors have thus shown that COMT inhibitors are capable of inhibiting the expression of genes involved in the osteoarthritis destruction process in articular chondrocytes, in particular the gene encoding a pro-inflammatory cytokine such as interleukin-6, and a metalloprotease such as MMP-13.

The inventors have thus demonstrated the direct inhibiting action of COMT inhibitors on several factors involved in cartilage destruction and inflammation during the osteoarthritis process.

Moreover, the inventors have shown that COMT inhibitors are also capable of stimulating the expression of genes involved in cartilage biosynthesis, such as COL2A1, which encodes constituents of type 2 collagen.

The inventors have thus shown that COMT inhibitors not only reduce the catabolism of cartilage, but also stimulate its anabolism. Importantly, the inventors have shown that COMT inhibitors administered intra-articularly are capable of slowing down the progression of osteoarthritic cartilage degradation and of reducing the pain felt by the individual.

They have also shown that COMT inhibitors administered in the joints are also capable of reducing the pain associated with osteoarthritis via a local and direct action in the joints, by a mechanism that does not involve the central or peripheral nervous system.

The use of these inhibitors thus represents a new treatment strategy for osteoarthritis. This strategy is all the more advantageous since several COMT inhibitors have already been approved for other therapeutic applications.

### First Subject of the Invention

According to a first subject, the present invention thus relates to a COMT inhibitor for use in the treatment of osteoarthritis.

### COMT Inhibitor

In the present invention, the term “COMT inhibitor” or more simply hereinbelow “inhibitor” refers to any compound that is capable of inhibiting COMT enzyme activity or COMT enzyme expression in articular chondrocytes. Preferably, the COMT inhibitor is also capable of inhibiting COMT enzyme activity or COMT enzyme expression in articular synoviocytes.

According to one embodiment, the COMT inhibitor is capable of slowing down or inhibiting the enzymatic activity of COMT.

Preferably, the inhibitor used according to the invention is a nitrocatechol derivative.

The term “nitrocatechol derivative” means a compound comprising a catechol part bearing a nitro group in the ortho- or para-position relative to one of the hydroxyl groups of the catechol part.

The inhibitor used according to the invention is preferably a nitrocatechol derivative chosen from the group consisting of tolcapone, entacapone, opicapone, nitecapone, nebicapone, and mixtures thereof.

The inhibitor used according to the invention may also be an antibody which blocks COMT activity.

The term “antibody” denotes an immunoglobulin molecule, i.e. a molecule which contains an antigen-binding site capable of immunospecifically binding to an antigen. In the present invention, the term “antibody” encompasses not only whole antibody molecules, but also antibody fragments.

In one embodiment, the antibody is an anti-COMT monoclonal antibody.

According to another embodiment, the inhibitor is capable of slowing down or inhibiting COMT expression. Such an inhibitor may be an interfering RNA or an antisense oligonucleotide complementary to the COMT messenger RNA sequence.

The RNA interference strategy is well known to those skilled in the art. This technique involves a process that occurs naturally in animal cells, notably comprising the reduction or silencing of targeted gene expression. The formation of double-stranded RNA leads to the specific degradation by the host cell machinery of transcripts complementary to this double-stranded RNA. Thus, by targeting COMT with RNA interference, it is possible to reduce the level of expression or totally suppress COMT gene expression in targeted cells.

A small interfering RNA (hereinbelow siRNA) is characterized in that it has a region with a double-stranded (ds) structure. Inhibition is specific to the target sequence, the nucleotide sequence of one strand of the ds region of the RNA comprising 15 to 25 nucleotides and thus being identical to the portion of the target gene. The nucleotide sequence of the other strand of the RNA ds region is complementary to that of the first strand and to the target gene portion. A person skilled in the art will know how to prepare siRNAs targeting COMT. In particular, numerous programs are available for designing siRNAs:
“siSearch Program” (Improved and automated prediction of effective siRNA”, Chalk A. M., Wahlesdelt C., and Sonnhammer E. L. L, Biochemical and Biophysical Research Communications, 2004).
“SiDirect” (Direct: highly effective, target-specific siRNA design software for mammalian RNA interference, Yuki Naito et al., Nucleic Acids Res., Vol. 32, No. Web Server issue (C) Oxford University Press 2004).

According to one embodiment, the inhibitor comprises a double-stranded RNA of about 15 to 30 nucleotides, preferably 19 to 25, even more preferably 19, 20, 21, 22 or 23 nucleotides long complementary (strand 1) and identical (strand 2) to a part of the COMT sequence.

Preferably, the gene encoding COMT has a sequence with the GenBank database identification number NG_011526.

Preferably, COMT messenger RNA has a sequence with the GenBank database identification number NM_000754.

Preferably, the COMT protein has a sequence with the GenBank database identification number NP_000745 (MB-COMT isoform) or NP_009294 (S-COMT isoform).

These siRNAs may also include a TT or UU dinucleotide at the 3′ end.

In one particular embodiment, the nitrocatechol derivative, antibody, siRNA or antisense oligonucleotide described above is capable of specifically reducing or even blocking COMT expression. For example, the invention is directed toward a nitrocatechol derivative, a COMT-targeting antibody, a COMT-targeting siRNA or a COMT-targeting antisense oligonucleotide enabling a greater than 80%, 90%, 95% or 99% reduction in COMT expression, in which this ability is preferably tested in vitro on chondrocytes stimulated with interleukin-1β (IL-1β).

IL-1β is conventionally used to model osteoarthritis in vitro, and a person skilled in the art knows how to perform this type of test.

In a particular embodiment, the nitrocatechol derivative, antibody, siRNA or antisense oligonucleotide described above, in addition to its ability to reduce COMT expression as described above, is also capable of reducing the expression of matrix metalloprotease 13 (MMP13) and/or interleukin-6 (IL-6). For example, the invention is directed toward a nitrocatechol derivative, antibody, siRNA or antisense oligonucleotide enabling a reduction of at least 10%, 15%, 20%; 30%, 40% or 50% in the expression of MMP13 and/or IL-6, in which this ability is preferably tested in vitro on chondrocytes stimulated with interleukin-1β (IL-1β).

An antisense oligonucleotide (hereinbelow ASO) is a single-stranded DNA fragment that can bind specifically to a target messenger RNA. The nucleotide sequence of the antisense oligonucleotide is complementary to that of the messenger RNA it targets, and generally comprises between 15 and 25 nucleotides. By hybridizing to their target mRNA, ASOs induce its degradation by ribonuclease H (RNAse H).

The inhibitor used according to the invention is preferably chosen from the group consisting of tolcapone, entacapone, opicapone and mixtures thereof.

### Treatment Methods

The term “treatment” denotes any action that reduces, eliminates or delays the symptoms associated with a pathology in a subject to be treated. It comprises both curative and prophylactic treatment of a disease. A curative treatment is defined as a treatment leading to a cure or a treatment alleviating, improving and/or eliminating, reducing and/or stabilizing the symptoms of a disease or the suffering it causes. A prophylactic treatment comprises both a treatment leading to the prevention of a disease and a treatment reducing and/or delaying the incidence of a disease or the risk of its occurrence. In the context of the present invention, the term “treatment” refers more particularly to the slowing down or inhibition of cartilage destruction as part of the osteoarthritic process, accompanied by a reduction in joint pain.

Thus, the inhibitor used according to the invention enables the slowing down or inhibition of cartilage destruction in the context of the osteoarthritic process.

In the present invention, a “chondroprotector” or “basic osteoarthritis drug” denotes a drug which enables the process of destruction/degradation of cartilage to be slowed down or inhibited.

Advantageously, the inhibitor used according to the invention also allows a reduction in the pain caused by osteoarthritis, in particular without action on the central or peripheral nervous system. Advantageously, the inhibitor used according to the invention allows a reduction in the pain caused by osteoarthritis without direct action on the central nervous system.

Advantageously, the inhibitor used according to the invention may thus be used both as a chondroprotective agent and as a pain-relieving agent.

According to one embodiment, the inhibitor is used for the treatment of osteoarthritis, in which the treatment comprises slowing down the destruction of cartilage and reducing the pain associated with osteoarthritis. The treatment notably involves reducing the expression of genes involved in catabolism, such as MMP13, reducing the expression of genes involved in inflammation, such as IL-6, and stimulating the expression and secretion of collagen, such as type II collagen (COL2A1), in the joints.

As will be seen hereinbelow, the inhibitor according to the invention is preferably formulated so as to be administered directly to the joints, i.e. intra-articularly.

The present invention also relates to the use of an inhibitor according to the invention, preferably a nitrocatechol derivative chosen from the group consisting of tolcapone, entacapone, opicapone and mixtures thereof, for the manufacture of a medicament intended for the treatment of osteoarthritis, in which the medicament is preferably administered intra-articularly.

The present invention also relates to a method for treating osteoarthritis in a subject, said method involving administering a therapeutically effective dose of an inhibitor according to the invention to said subject, in which the inhibitor according to the invention is preferably a nitrocatechol derivative notably chosen from the group consisting of tolcapone, entacapone, opicapone and mixtures thereof, and in which the inhibitor according to the invention is preferably administered to said subject intra-articularly.

The present invention also relates to the use of an inhibitor according to the invention for the manufacture of a medicament intended for treating osteoarthritis, in which the inhibitor according to the invention is preferably a nitrocatechol derivative notably chosen from the group consisting of tolcapone, entacapone, opicapone and mixtures thereof, and in which the inhibitor according to the invention is preferably administered to said subject intra-articularly.

### Osteoarthritis

For the purposes of the present invention, the term “osteoarthritis” refers to any chronic articular pathology resulting in structural deterioration of articular cartilage. The term “osteoarthritis” thus notably includes primary or primitive osteoarthritis, in which the subject suffers from osteoarthritis without any identifiable cause or particular predisposition, in particular without any traumatic cause or associated inflammatory disease. The term “osteoarthritis” also includes secondary osteoarthritis, in which the subject has suffered trauma to a joint (in which case the osteoarthritis is referred to as “traumatic osteoarthritis”), for example in a road accident, or suffers from a disease predisposing to osteoarthritis, for example chronic inflammatory rheumatism such as rheumatoid arthritis, spondyloarthropathy, microcrystalline rheumatism, or metabolic diseases such as obesity, diabetes or hemochromatosis. More generally, in the present invention, the term “osteoarthritis” also comprises all joint pathologies in which structural deterioration of articular cartilage is observed, such as rheumatoid arthritis, gout or psoriatic arthritis, as well as diseases which affect the joints and generate pain, such as hemochromatosis, joint dysplasias and rare skeletal diseases.

Suspicion of osteoarthritis is based on questioning and clinical examination. The patient describes mechanical joint pain, with precise localization. Pain is usually reproduced on clinical examination.

The diagnosis of osteoarthritis is based on imaging, in particular standard X-rays, CT scans and MRI.

Standard X-rays make it possible to identify the main feature of osteoarthritis, namely the reduction in cartilage quantity (the most commonly used radiological term is joint pinch), and, depending on the stage of osteoarthritis, bone damage, such as osteosclerosis (hardening of the bone immediately beneath the cartilage, which looks whitened on X-ray), the presence of osteophytes (bony outgrowths on X-ray), and/or geodes (cysts in the bone beneath the cartilage, which are visible on X-ray).

A radiological definition is based on the Kellgren-Lawrence classification (Kellgren J. H., Lawrence J. S. Radiological assessment of osteoarthritis. Ann. Rheum. Dis., 1957, 16: 494-502). The Kellgren-Lawrence score is a composite index that notably takes into account osteophytes and joint space pinching. This classification includes four stages: doubtful, minimal, certain and advanced osteoarthritis. Stage 2 is usually the diagnostic threshold. The radiological stages of knee osteoarthritis (gonarthrosis) are as follows: Stage 0 normal radiograph; Stage 1 osteophyte of doubtful significance; Stage 2 clear osteophyte with no change in the joint space; Stage 3 clear osteophyte and reduced joint space; Stage 4 severe pinching of the joint space and sclerosis of the subchondral bone.

The radiological stages of hip osteoarthritis (coxarthrosis) according to the Kellgren-Laurence classification are as follows: Stage 0: normal radiograph; Stage 1: joint pinch, doubtful pericapital osteophytosis; Stage 2: joint pinch, osteophytosis, moderate bone sclerosis; Stage 3: clear joint pinch with discrete osteophytosis, bone sclerosis with cyst, minimal deformity of femoral head and acetabulum; Stage 4: disappearance of joint space with bone sclerosis and cyst, significant deformity of femoral head and acetabulum, with major osteophytosis.

CT or MRI scans are advantageous, and are often performed in cases where clinical symptoms are strongly suggestive of osteoarthritis, when X-rays are normal. These imaging tests are more sensitive and allow the detection of earlier abnormalities not visible on standard X-rays.

In the present invention, osteoarthritis may affect any joint, notably those of the spine and peripheral joints, in particular those of the knee (gonarthrosis), hip (coxarthrosis), ankle, foot, hand, wrist, elbow or shoulder.

According to one embodiment, the osteoarthritis to be treated is osteoarthritis of a peripheral joint, preferably osteoarthritis of the knee.

According to one embodiment, the osteoarthritis to be treated is a primary or secondary osteoarthritis, preferably a primary osteoarthritis.

The inhibitor according to the invention is preferably used in the treatment of osteoarthritis of a peripheral joint, in particular a knee joint.

According to one embodiment, the inhibitor according to the invention is used in the treatment of osteoarthritis which is not primary osteoarthritis.

### Subject to be Treated

The subject to be treated, or patient, is an animal, preferably a mammal.

According to one embodiment, the subject to be treated is a human being.

The human subject is preferably an adult aged over 40, preferably over 50.

The subject is preferably one having stage 2 or 3 osteoarthritis according to the Kellgren-Lawrence classification.

According to another embodiment, the subject is a domestic animal, preferably a pet, an animal intended for sporting use, or a production animal.

The pet is preferably chosen from a cat or a dog.

The animal intended for sporting use is preferably a horse, typically an equestrian horse.

The production animal is preferably chosen from a bovine such as a cow, bull or ox, an ovine such as a sheep, or a pig.

According to one embodiment, the subject to be treated has no central and/or peripheral nervous system disorders, such as Parkinson's disease, schizophrenia and other dopamine-deficiency diseases.

According to one embodiment, the subject to be treated has no pain other than that associated with osteoarthritis.

### Administration Method

The inhibitor used according to the invention may be administered via any known route, notably intra-articular, oral or subcutaneous.

According to a particularly preferred embodiment, the inhibitor is administered by intra-articular injection, in particular into the arthritic joint.

The term “intra-articular”, relating to the administration method, denotes any route of administration intended for the implantation or injection of a compound or composition within or close to the intra-articular space of a joint, in particular an arthritic joint. Preferably, the term “intra-articular” denotes an administration method in which the inhibitor is administered into the intra-articular space of an arthritic joint.

The inventors have shown that, particularly surprisingly, intra-articular administration of a COMT inhibitor allows the progression of cartilage degradation to be limited.

In an entirely surprising manner, the inventors have shown that intra-articular administration of a COMT inhibitor can also sustainably reduce the subject's pain, even when the COMT inhibitor is administered locally and not systemically.

The inventors consider that intra-articular administration of the inhibitor according to the invention has the advantage of maximizing the inhibitor's efficacy both in limiting cartilage degradation and in reducing pain. In other words, without wishing to be bound by any particular theory, the inventors consider that intra-articular administration of the inhibitor according to the invention has the advantage of reducing the therapeutically effective dose relative to other routes of administration, such as the oral route. Also, as mentioned previously, the inventors consider that the inhibitor administered intra-articularly allows pain to be reduced by a local and direct mechanism in the joints, which does not involve the central or peripheral nervous system.

In the case of intra-articular administration, as will be detailed hereinbelow, the inhibitor according to the invention may be administered in combination with other substances, in particular substances enabling sustained release of the inhibitor, such as the use of hydrogels, for example of hyaluronic acid.

The inhibitor used according to the invention can also be administered orally. Oral administration is particularly preferred when the subject is a domestic animal, in particular a cat, dog or horse. According to one embodiment, the subject is a domestic animal and administration is intra-articular.

The inhibitor used according to the invention is preferably administered to the subject in a therapeutically effective dose. The term “therapeutically effective dose” as used herein refers to the amount necessary to observe a therapeutic or preventive activity on osteoarthritis, in particular the amount necessary to observe an inhibition or a slowing down of the destruction of osteoarthritic cartilage. The amount of inhibitor to be administered, and also the duration of treatment, may be assessed by a person skilled in the art on the basis of criteria such as the physiological state, sex, age and weight of the subject to be treated, the nature of the osteoarthritic joint(s) to be treated, the inhibitor chosen, and the route of administration used. The therapeutically effective dose for each treatment may be administered in the form of a single dose or in portionwise doses.

The inhibitor used according to the invention may be administered in the form of a single dose or in portionwise doses.

According to one embodiment, the inhibitor used according to the invention is preferably a nitrocatechol derivative chosen from the group consisting of tolcapone, entacapone, opicapone and mixtures thereof, and administered to the subject intra-articularly.

According to one embodiment, the inhibitor used according to the invention is preferably a nitrocatechol derivative chosen from the group consisting of tolcapone, entacapone, opicapone and mixtures thereof, and administered to the subject orally.

The inhibitor used according to the invention may be administered in combination with other active substances. The inhibitor according to the invention and the other active substance(s) may be administered simultaneously (at the same time) or sequentially (at different times), via the same or different routes of administration.

In the context of the present invention, the active substances useful in the treatment of osteoarthritis notably include:
symptomatic rapid-acting drugs for osteoarthritis, which have efficacy in treating the painful symptoms of osteoarthritis. These include various products, among which are analgesics such as paracetamol, non-steroidal anti-inflammatory drugs such as aceclofenac, niflumic acid, tiaprofenic acid, celecoxib, diclofenac, etodolac, etoricoxib, flurbiprofen, ibuprofen, indomethacin, ketoprofen, meloxicam, nabumetone, naproxen, piroxicam, sulindac tenoxicam, and steroidal anti-inflammatory drugs notably of the corticoid type, in particular injectable corticoids with immediate or sustained action, such as betamethasone;
symptomatic slow-acting drugs for osteoarthritis, or SYSADOAs, which have an efficacy on the painful symptoms of osteoarthritis. These include various products, among which are platelet-rich plasma (PRP), chondroitin, for example chondroitin sulfate, glucosamine, for example glucosamine phosphate or sulfate, and hyaluronic acid. Glucosamine and chondroitin are usually administered orally. Hyaluronic acid and PRP are usually administered intra-articularly;
chondroprotectors or basic osteoarthritis drugs. As defined above, chondroprotectors have an efficacy in slowing down or inhibiting the process of cartilage destruction/degradation. Certain SYSADOAs, such as PRP, are sometimes considered to have chondroprotective activity; however, this remains debated.

Thus, according to one embodiment, the inhibitor used according to the invention can notably be administered in combination with one or more active substances chosen from the group consisting of analgesics such as paracetamol, non-steroidal anti-inflammatory drugs such as aceclofenac, niflumic acid, tiaprofenic acid, celecoxib, diclofenac, etodolac, etoricoxib, flurbiprofen, ibuprofen, indomethacin, ketoprofen, meloxicam, nabumetone, naproxen, piroxicam, sulindac tenoxicam; steroidal anti-inflammatory drugs, particularly of the corticoid type, in particular injectable corticoids with immediate or sustained action, such as betamethasone; symptomatic slow-acting drugs for osteoarthritis, such as platelet-rich plasma (PRP), chondroitin, for example chondroitin sulfate, and glucosamine, for example glucosamine phosphate or sulfate; chondroprotectors.

### Second Subject of the Invention

The inhibitor of the present description is preferably administered in the form of a pharmaceutical composition.

Thus, according to a second subject, the invention relates to a pharmaceutical composition comprising one or more COMT inhibitors according to the first subject of the invention, for the treatment of osteoarthritis, and a pharmaceutically acceptable excipient.

The term “pharmaceutically acceptable” refers to a substance which is not biologically or otherwise undesirable, i.e. which can be incorporated into a pharmaceutical composition administered to a patient or animal without causing adverse biological effects or without adversely interacting with any of the other components of the composition in which it is contained, for example by inhibiting or reducing the anti-inflammatory properties of the COMT inhibitor.

Typically, the pharmaceutically acceptable excipient may be chosen from a diluent, disintegrant, binder, glidant, lubricant, wetting agent, buffering agent, suspending agent, adjuvant, emulsifier, absorbent, preserving agent, surfactant, sweetening agent, antioxidant, or a mixture thereof. These excipients are described, for example, in “The Science and Practice of Pharmacy 1995, edited by E. W. Martin, Mack Publishing Company, 19th edition, Easton, Pa”.

The amount of COMT inhibitor according to the invention in the compositions may be varied so as to administer an effective amount of inhibitor to obtain the desired therapeutic response for a particular patient or pet.

The composition may also comprise one or more other active substances as defined above, preferably chosen from the group of injectable immediate or sustained-action corticoids such as betamethasone; symptomatic slow-acting drugs for osteoarthritis such as platelet-rich plasma (PRP), chondroitin, for example chondroitin sulfate, and glucosamine, for example glucosamine phosphate or sulfate; chondroprotectors.

The inhibitor(s) are preferably one or more nitrocatechol derivatives chosen from the group consisting of tolcapone, entacapone, opicapone and mixtures thereof.

The pharmaceutical composition used according to the invention is preferably in the form of an ingestible or injectable composition, preferably in the form of a composition intended for intra-articular or oral administration.

In one embodiment, the pharmaceutical composition used according to the invention is a composition in the form of an injectable composition intended for intra-articular administration.

In this embodiment, the composition has the advantage of being in the form of a sustained-release composition.

The term “sustained-release”, in relation to the formulation, denotes a formulation allowing the clearance of the inhibitor according to the invention to be reduced, or in other words to increase the half-life of the inhibitor in the joint after administration. The terms “sustained-release”, “controlled-release”, “delayed-release” and “long-acting” are used interchangeably in the present invention.

A sustained-release pharmaceutical composition has the advantage of increasing the therapeutic efficacy of each dose, and, in the context of portionwise administration, of lengthening the time interval between two administrations.

In this embodiment, the pharmaceutical composition used according to the invention may notably comprise the inhibitor according to the invention and a polymeric fraction, the inhibitor preferably being dispersed in the polymeric fraction.

The polymeric fraction may notably comprise a polymer, preferably a biodegradable and/or biocompatible polymer, chosen from the group including but not limited to hyaluronic acid, collagen, chitosan, polylactic acid (PLA), glycolic acid, copolymers of lactic acid and glycolic acid (PLGA), polyoxalates, polycaprolactone, polyesters, and mixtures thereof.

In one embodiment, the polymeric fraction is in the form of a gel or is capable of forming a gel.

The term “gel” denotes a non-flowing polymer network swollen with a solvent.

The expression “polymeric fraction that is capable of forming a gel” refers to a polymeric fraction that is capable of gelling in situ, i.e. forming a gel once placed in the biological environment in which it has been administered, typically the intra-articular space.

The pharmaceutical composition in this embodiment may, for example, be in the form of a solution comprising the inhibitor(s) and the polymeric fraction, or a suspension of particles, typically microparticles, in which the particles are formed from the polymeric fraction in the form of a gel in which the inhibitor(s) are encapsulated.

In a particular embodiment, the polymer, polymeric fraction or gel is biocompatible and/or biodegradable.

The term “biodegradable”, relative to the polymer, polymeric fraction or gel, means that, in situ, i.e. in the joint, the polymer, polymeric fraction or gel is capable of degrading naturally, under physiological conditions, thus allowing sustained release of the inhibitor. The reactions involved in biodegradation may include hydrolysis reactions, i.e. the breaking of covalent bonds by reaction with water. These reactions may be catalyzed by the action of enzymes naturally present at the injection site, typically in the joint.

According to the present disclosure, the term “biocompatible” relating to the polymer, polymeric fraction or gel, means a polymer, polymeric fraction or gel having the capacity of not degrading the biological environment in which it is placed.

According to one embodiment, the pharmaceutical composition used according to the invention comprises one or more inhibitors according to the first subject of the invention, preferably one or more nitrocatechol derivatives chosen from the group consisting of tolcapone, entacapone, opicapone and mixtures thereof, and hyaluronic acid.

### EXAMPLES

### Example 1: In Vitro Tests on Human Cartilage (Tolcapone)

### Material and Methods:

### Cell Culture:

The cells were obtained from surgical waste in accordance with current regulations (protocol approved by the Comité de Protection des Personnes (CPP) Nord-Ouest III). After patients have signed a free and informed consent form, femoral heads, synovial membranes and bone marrow are recovered on conclusion of hip arthroplasty operations on osteoarthritic patients.

The femoral heads are dissected and cartilage chips are produced. The chondrocytes are released by digestion with pronase type XIV (2 mg/ml for 15 minutes; Sigma), followed by digestion with collagenase type II (2 mg/ml overnight; Gibco). The cells are then seeded at 4×10⁴ cells/cm² in Dulbecco's modified Eagle's medium (DMEM) supplemented with 10% fetal calf serum (FCS) and an antibiotic cocktail. They are then incubated at 37° C. in a humidified atmosphere containing 5% CO₂.

Synoviocytes are obtained from synovial membranes of patients undergoing hip replacement surgery. The synoviocytes are released by digestion with type I collagenase (2 mg/mL overnight; Gibco). After rinsing with PBS, the recovered cells are seeded with DMEM medium supplemented with 10% FCS and an antibiotic cocktail.

Bone marrow cells were fractionated on a Hypaque-Ficoll density gradient. Mononuclear cells were isolated, seeded at a density of 5×10⁴ cells/cm² and cultured in alpha-MEM supplemented with 10% fetal calf serum (Invitrogen, Cergy-Pontoise, France), 2 mM L-glutamine, 1 ng/ml FGF-2 (Sigma Chemical Co.) and antibiotics. At nearly 80% confluence, cells were harvested by trypsinization (0.25% trypsin/1 mM EDTA, Invitrogen) and seeded at 10³ cells/cm². After five passages, disappearance of the hematopoietic markers CD34 and CD45 was verified by RT-PCR and the cells were used for the experiments.

For all three cell types, the cells were amplified in a humid chamber at 37° C. under 5% CO₂. The culture media were changed two to three times a week. A mycoplasma test was performed so as to check that they were not contaminated. A portion was treated with 1 ng/mL IL-1p (Sigma) diluted in DMEM medium with 10% FCS and 0.1% penicillin and streptomycin for 48h, in the presence or absence of tolcapone (HY-17406, CliniSciences).

mRNA Extraction and RT-PCR:

Total RNAs are extracted using the RNeasy mini kit (Qiagen) according to the manufacturer's instructions. 1 μg of RNA is then treated with DNAse I (Sigma-Aldrich) and then reverse transcribed into cDNA using Moloney murine leukemia virus reverse transcriptase (Invitrogen) according to the supplier's protocol. The cDNAs are diluted 1:100 and stored at −20° C. until the time of use in real-time PCR.

Real-time PCR primers used routinely in the laboratory or designed using the Primer-BLAST tool (NCBI) against RPL13A, IL6, MMP13 and COMT are used.

The sense and antisense primers are diluted to 130 nM, with 5 μl of 100-fold diluted cDNA and also 7.5 μl of Power SYBR Green master mix in a reaction volume of 15 μL (Applied Biosystems). The samples are denatured at 95° C. for 10 seconds, then primer hybridization and polymerization are performed for 1 minute at 60° C. for 40 cycles. These cycles are performed using the Step One Plus Real Time PCR system (Applied Biosystems). Relative mRNA expression levels were calculated using the 2-ΔΔCT method and normalized to the expression of RPL13A, a housekeeping gene conventionally used as a reference for normalizing gene expression in human joint cells. In order to visualize amplification, samples after RT-PCR were plated on a 2% agarose gel. Signals were revealed using a Bio-Rad imager.

### Protein Extraction and Western-Blot:

After treatment, the cell lawns are rinsed with PBS, and then scraped in a lysis buffer known as “radioimmunoprecipitation” (RIPA: Tris-HCl 50 mM; IGEPAL 1%; NaCl 150 mM; EGTA 1 mM; NaF 1 mM) supplemented with protease inhibitors (leupeptin, aprotinin, pepstatin, phenylmethylsulfonyl fluoride (PMSF), and phosphatases (sodium orthovanadate Na₃VO₄), so as to lyze the cells and extract the protein. The cell lyzates obtained are incubated for 30 minutes at 4° C., and then centrifuged for 30 minutes at 10 000×g at 4° C. The protein-containing supernatants are recovered and stored at −20° C. until the time of use in Western blotting. The protein concentration of each sample is then determined according to the Bradford method with the Bradford Protein Assay reagent (Biorad). A calibration range is produced in duplicate and the samples in triplicate.

30 μg of protein per sample migrate by electrophoresis on a sodium dodecyl sulfate-polyacrylamide gel (SDS-PAGE) and are transferred onto polyvinylidene difluoride membranes (Bio-Rad). After blocking the nonspecific sites, the membrane is incubated with 1000-fold diluted anti-COMT primary antibodies (PA5-76864; Thermo-Fisher) overnight at 4° C. After rinsing, the membranes are incubated with peroxidase-coupled secondary antibodies (SC-516087, Santa-Cruz) for 1 h at room temperature. After incubation with the oxidizing agent and luminol (1 minute with stirring, Western Lightning ECL Pro, PerkinElmer. Actin (sc-47778, Santa-Cruz) was used in order to check the homogeneous deposition of the samples. Signals were revealed using an imager (Bio-Rad).

### Proteomics:

Proteomics experiments were performed in the form of a service on the Proteogen platform at the University of Caen Normandie. Analyses were performed using protein samples from the cell lawn (RIPA extraction) or directly on culture media, as described in the following article (Brochard S., Pontin J., Bernay B., Boumediene K., Conrozier T., Baugé C. The benefit of combining curcumin, bromelain and harpagophytum to reduce inflammation in osteoarthritic synovial cells. BMC Complement Med. Ther. 2021 Oct. 14; 21(1):261. doi: 10.1186/s12906-021-03435-7. PMID: 34649531; PMCID: PMC8515758.).

### Results:

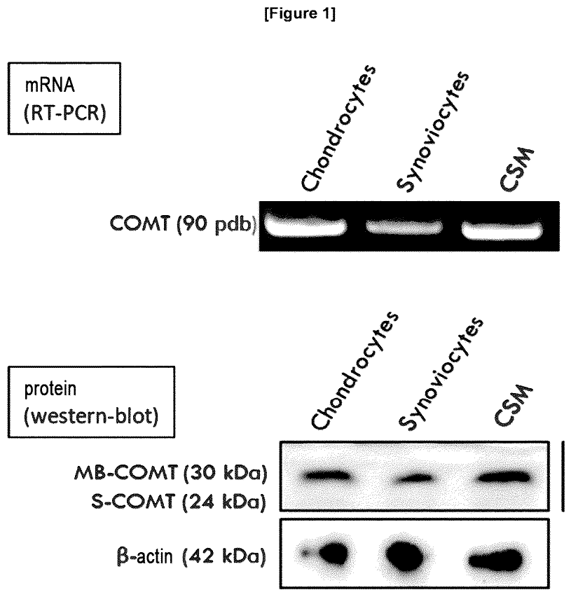

COMT expression in different cell types present in joints (chondrocytes, synoviocytes, mesenchymal stem cells) is analyzed by RT-PCR and Western-blot (FIG. 1). These analyses revealed that COMT is expressed at both mRNA and protein levels in the three cell types studied. Thus, the RT-PCR results show that human chondrocytes, human synoviocytes and human bone marrow-derived mesenchymal stem cells express COMT mRNA. Moreover, Western-blot analysis showed that all three cell types produced both soluble (24 kDa) and membrane-bound (30 kDa) forms of COMT protein.

Having demonstrated that COMT was expressed in chondrocytes, a study was performed on the effect of inhibiting COMT activity in these cells when stimulated with interleukin-1β (IL-1β). IL-1β is indeed conventionally used to model osteoarthritis in in vitro studies. This pro-inflammatory cytokine is present in osteoarthritic joints and is involved in the pathophysiology of osteoarthritis (inflammation, catabolism). Thus, human articular chondrocytes were treated with increasing doses of a pharmacological COMT inhibitor, tolcapone.

The experiments herein (FIG. 3) clearly show that COMT inhibition with tolcapone counteracts the damaging action of IL-1β and notably reduces the expression of genes involved in catabolism (such as MMP13) and inflammation (such as IL-6) in chondrocytes.

In order to have a more comprehensive view of the action of COMT inhibitors on chondrocyte phenotype, proteomics experiments were performed. The results of these experiments (not shown) show that pharmacological inhibition of COMT affects the expression of a large number of proteins involved in the osteoarthritic process in human articular chondrocytes. Pharmacological inhibition of COMT notably stimulates the expression and secretion of collagens, notably type 2 collagen (COL2A1).

In particular, the following were observed in human articular chondrocytes:
reduced MMP13 and IL-6 secretion in the presence of tolcapone, confirming the anti-catabolic and anti-inflammatory effects of tolcapone,
an increase in COL2A1 secretion, suggesting an anabolic effect of tolcapone.

These results are shown in the table below:

TABLE 1
Secretome study: comparison of proteins secreted by IL1-stimulated
chondrocytes in the presence or absence of tolcapone.

| | | |
|---|---|---|
| Gene | logFC | p-value |

Proteins whose expression is inhibited with tolcapone

| | | |
|---|---|---|
| MMP13 | −1.78702 | 0.0227957 |
| IL6 | −2.56409 | 0.0286182 |

Proteins whose secretion is increased with tolcapone

| | | |
|---|---|---|
| COL2A1 | 4.51503 | 0.0386184 |

These results confirm the anti-inflammatory and anti-catabolic action of COMT inhibition in IL-1-stimulated human articular chondrocytes, and moreover show a complementary action on anabolism, since an increase in the expression and secretion of type 2 collagen protein (an essential matrix protein in cartilage) was identified. These results show that targeting COMT may have an anti-catabolic, anti-inflammatory and chondroprotective action, and suggest a direct action on the cells present in the joints.

### Example 2: In Vivo Tests in Mouse Models

### Material and Methods:

### Animals, Osteoarthritis Induction and Treatments

This study was authorized by the French Ministry of Higher Education, Research and Innovation, following a favorable opinion from the local ethics committee, CENOMEXA (approval no. B14118015). The study was performed in an approved animal facility (UMS-CYCERON). 10-week-old male C57BL7 mice were used. The mice were placed 8 to a cage, in reverse cycle, with food and water available ad libidum. The health status of each mouse was evaluated daily.

The traumatic osteoarthritis model, reproduced by destabilization of the mouse internal meniscus (DMM), was induced under general anesthesia. After disinfecting the skin with 700 alcohol, the skin and subcutaneous fascia were cut with sterile scissors. Next, the medial collateral ligament was cut transversely, and the fibers of the vastus medialis muscle were sectioned longitudinally to allow external dislocation of the patella. Meniscectomy of the internal meniscus of the right knee was then performed under a magnifying glass. Finally, the vastus medialis muscle and skin were sutured. These mice constituted an osteoarthritis group (DMM). A control group, known as the “sham” group, consisting of mice undergoing the same procedure without meniscectomy, was established.

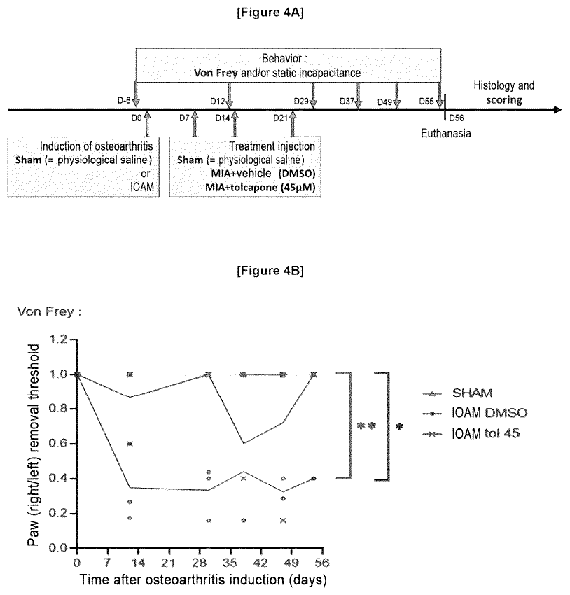

For the inflammatory osteoarthritis model (IOAM), intra-articular injection with monosodium iodoacetate (0.75 mg in 10 μl) was performed under general anesthesia. After clinically locating the patellar ligament, the injections were made intra-articularly into the knee after passing through the patellar ligament with a 27 Gauge needle. These mice constituted an osteoarthritis group (IOAM). A control group, known as the “sham” group, consisting of mice undergoing injection with saline, is constituted. Tolcapone injections were performed using the same protocol at 7, 14 and 21 days after osteoarthritis induction, according to the experimental scheme represented in FIG. 4A.

### Immunohistology

Mouse knees were fixed in 4% PFA for 24 h and decalcified in 0.5 M EDTA pH 8.0 for 10 days before embedding in paraffin. Sagittal sections 5 μm thick were produced and mounted on Silanized SuperFrost® microscope slides. Before use, the slides were deparaffinized in three xylene baths for 15 min each. They were then rehydrated in successive baths of 100%, 70% and 40% ethanol, and then two successive baths of water and PBS. Antigenic unmasking is performed by heat (70°) in citrate buffer (pH 6) for 4h, followed by hyaluronidase digestion (1 mg/ml in PBS). After two rinses in PBS, the endogenous peroxidases are inhibited by incubation with 3% H₂O₂ at room temperature for 10 minutes. The nonspecific sites are blocked by incubation at room temperature for 30 minutes with a blocking solution (2.5% horse serum, 2.5% BSA in PBS). The rabbit anti-COMT polyclonal antibody (PA5-76864; Thermo-Fisher) is used diluted 500-fold in half-strength blocking solution, incubated overnight at 4° C. Detection is performed by means of the ImmPRESS staining reagent kit (Vector Laboratories, France) with an anti-rabbit antibody. Revelation is performed using the diaminobenzidine substrate kit for peroxidase (Vector Laboratories, France). Finally, counter-staining with blue water (three drops of 1% toluidine blue in 200 ml water) before mounting with ENTELAN.

### Histology

The joints are placed in a fixation solution (37% formaldehyde, PBS) for 3 days, and then decalcified using Osteosoft for 48 h. The joints are then stored at −80° C. in Optimal Cutting Temperature compound (OCT). 10 μm-thick sections were produced at −25° C. using a Cryostat (CM 3050 S, Leica BIOSYSTEMS, France), and then mounted on SuperFrost® microscope slides. The slides are then stained with Fast Green and then Safranin-O. Safranin-O permits revelation of articular cartilage components. Glycosaminoglycans and proteoglycans are seen in red. Fast Green acts as a counter stain, coloring the mineralized bone green.

### Von Frey:

Mechanical allodynia (pain triggered by a stimulus that is normally painless) is evaluated by the Von Frey filament test (BIOSEB In Vivo Research Instruments®). This test is based on the principle that when the end of a filament (of given length and diameter) is applied perpendicularly, the force exerted by the filament increases until the filament flexes. Beyond this point, the force exerted by the filament remains constant. For this test, the mice are placed in an individual cage, on a grid. A 30-minute habituation period was required before the test began. Filaments exerting progressively greater force were applied under the mouse's paw, from bottom to top, perpendicular to the mouse's plantar surface, until the filament began to flex. Withdrawal of the paw, startle or licking of the paw are interpreted as positive responses, synonymous with pain or discomfort. The results are presented as the value obtained on the arthritic paw, relative to the sensitivity of the contralateral (non-arthritic) paw.

### Results:

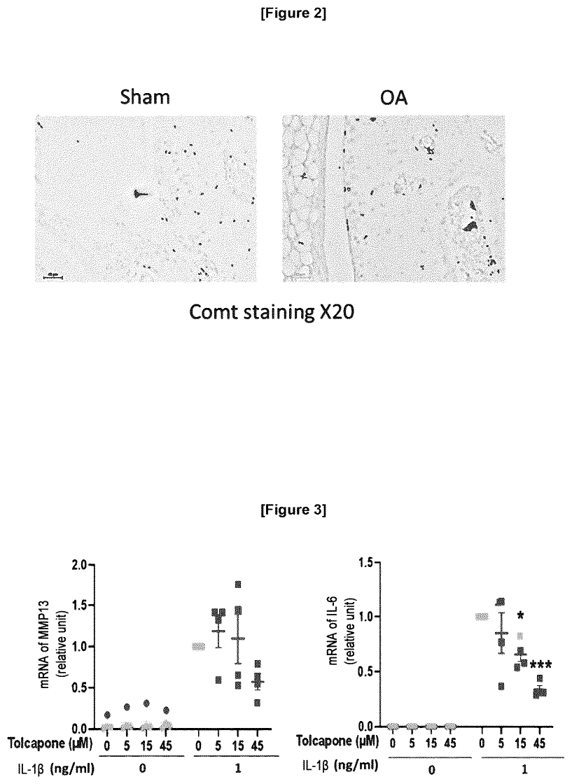

This study was continued by investigating the protein expression of COMT in the joint of normal and osteoarthritic mice (DMM trauma model) (FIG. 2). Immunohistochemistry results show that COMT expression is increased in the osteoarthritic joint compared to a healthy joint, and that COMT is mainly expressed by chondrocytes.

The effect of tolcapone was then tested in vivo. To this end, intra-articular injections of tolcapone were performed 7 days, 14 days and 21 days after the induction of osteoarthritis in mice (inflammatory model IOAM). Pain was evaluated throughout the experiment by Von Frey analysis. Histological studies were performed at 8 weeks (see experimental design, FIG. 4A).

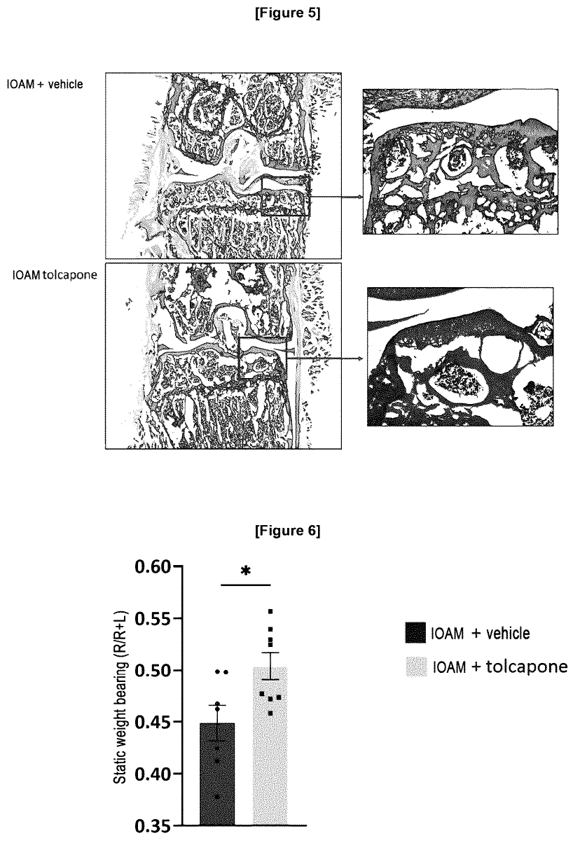

The results herein clearly show a beneficial effect of tolcapone on the progression of osteoarthritis. On the one hand, it is observed that intra-articular injection of tolcapone into the osteoarthritic joint markedly reduces induced pain (FIG. 4B), while at the same time improving joint structure (FIG. 5). Thus, the histological study clearly shows that intra-articular injections of the COMT inhibitor into the osteoarthritic joint slowed the progression of osteoarthritis as it reduced joint damage and very strongly limited cartilage degradation in these mice (“IOAM+tolcapone”) compared to untreated osteoarthritic mice (“IOAM+vehicle”).

These results demonstrate that COMT inhibition in the joints is useful in treating osteoarthritis. Not only does it slow the progression of osteoarthritis (chondroprotective effect), it also simultaneously reduces the pain induced.

### Example 3: In Vivo Test in Mouse Model: Additional Characterizations (OARSI/SWB)

The study of Example 2 was continued by investigating the effects of tolcapone on static disability (posture test) and OARSI score (quantification of osteoarthritis progression).

### Materials and Methods

### Animals, Osteoarthritis Induction and Treatments

The animals, osteoarthritis induction and treatments are as described in Example 2.

### Static Incapacitation

The static weight bearing (SWB; BioSeb, France) test allows evaluation of the weight applied by the animal under each hind leg in a static position. To do this, the animal is placed in a transparent restraint box so that its hind legs rest on two weight sensors connected to a recorder. This recorder is connected to a foot pedal allowing acquisition to begin. Zeroing is performed between each animal and verified using a 2 g calibration weight. The recording parameters are 2 seconds per acquisition. The value retained is an average of the weight applied during these 2 seconds. Ten successive acquisitions are performed. Acquisition values are retained only if the animal has not changed position during recording, and if its hind legs are the only elements resting on the sensors. For analysis, the average weight applied under the right paw is referred to the average weight applied on both paws (right+left).

### OARSI Score:

Joint specimens after staining with safranin-O were scored using the Osteoarthritis Research Society International (OARSI) scale as detailed in Pritzker et al., 2006 (Pritzker, K. P., et al., Osteoarthritis cartilage histopathology: grading and staging. Osteoarthritis Cartilage, 2006. 14(1): pages 13-29). This score is divided into six grades, the last two of which include changes in the subchondral bone. The different grades of the Pritzker OARSI score allow precise quantification of cartilage degradation, with scores ranging from 0 to 6.5. The higher the score, the greater the degree of osteoarthritis.

### Results

### Static Incapacitation

The results are shown in FIG. 6.

The results clearly show a beneficial effect of tolcapone on static weight bearing. These results show that tolcapone allows the animal's posture to be restored, i.e. similar support on both hind legs (no difference in support between the arthritic leg and the normal leg), confirming the absence of pain in the arthritic leg after tolcapone treatment.

### OARSI Score

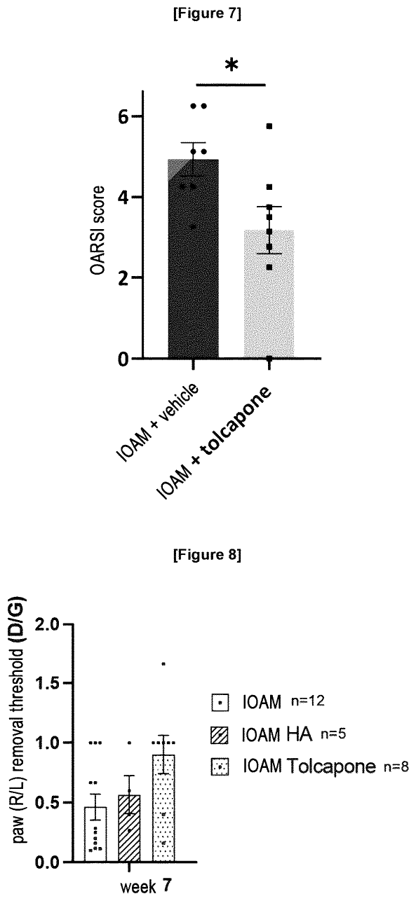

The results are shown in FIG. 7.

The results show a beneficial effect of tolcapone on the progression of osteoarthritis.

### Example 4: In Vivo Test in Mouse Model: Tolcapone/Hyaluronic Acid Comparison

The study presented in Example 2 is continued by comparing the effects of tolcapone with those of hyaluronic acid (HA).

### Material and Methods:

### Animals, Osteoarthritis Induction and Treatments

The animals, osteoarthritis induction and treatments are as described in Example 2, except that three groups of mice are tested:
IOAM: IOAM mice subjected to saline injections,
IOAM HA: IOAM mice subjected to hyaluronic acid injections
IOAM Tolcapone: IOAM mice subjected to tolcapone injections.

### Von Frey:

Pain is evaluated by means of the Von Frey technique as detailed in Example 2, 7 weeks after injection.

### Results:

The results are shown in FIG. 8.

The results show that intra-articular injection of tolcapone into the osteoarthritic joint reduces induced pain at least as much as or even more than hyaluronic acid.

### Example 5: In Vitro Test on Human Cartilage (Synoviocytes)

The study of Example 1 was continued by investigating the effect of tolcapone on synoviocytes.

### Material and Methods:

The materials and methods are the same as those described in Example 1, except that the study is performed on synoviocytes.

### Results:

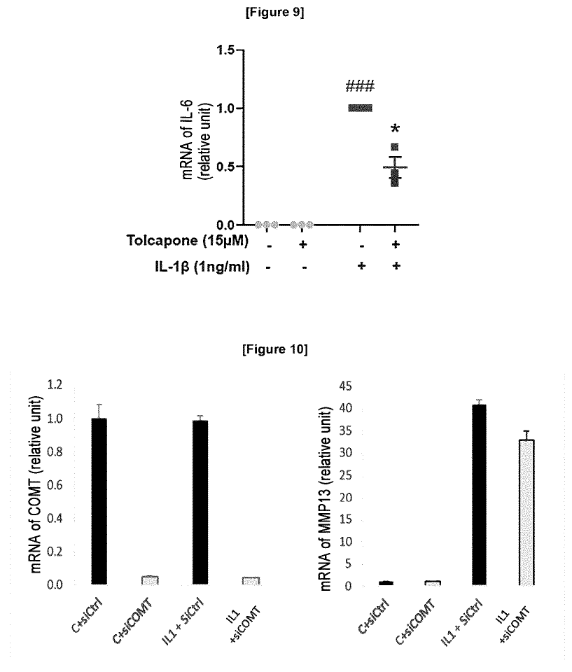

The results are shown in FIG. 9.

The results show that, similarly to articular chondrocytes, pharmacological inhibition of COMT with tolcapone reduces the expression of the genes involved in the osteoarthritic process (inflammation) in human articular synoviocytes, such as IL-6.

### Example 6: In Vitro Test on Human Cartilage (SiRNA)

The study of Example 1 was continued by investigating the effect of siRNAs directed against COMT.

### Material and Methods:

The materials and methods are the same as those described in Example 1, except that the cells are placed in the presence of SiRNA.

Control siRNAs or siRNAs directed against COMT (ON-TARGETplus Human COMT (1312) siRNA—SMARTpool, Dharmacon) were transfected into chondrocytes by nucleofection (Amaxa), following the manufacturer's protocol. In short, chondrocytes were harvested. Then, 4 million cells were mixed with 200 nmol of siRNA (si-control or si-COMT) in P3 Primary Cell 4D-Nucleofector X solution and placed in 1 cm² transfection cuvettes for electroporation using the 4D-Nucleofector X unit. The predefined ER-100 program was used. After nucleofection, the cells were plated out in appropriate dishes and incubated in culture medium.

### Results:

The results are shown in FIG. 10.

The results show that COMT knockdown by RNA interference reduces MMP13 expression in IL1-stimulated chondrocytes. This shows that pharmacological inhibition of COMT reduces the expression of the genes involved in the osteoarthritic process (catabolism) in human articular chondrocytes, whatever the structure of the COMT inhibitor used: nitrocatechol derivative such as tolcapone (cf. Example 1) or small interfering RNA as in the present example.

1. A method of treating a subject for osteoarthritis comprising providing the subject with a catechol O-methyltransferase (COMT) inhibitor.

2. The method claimed in claim 1, wherein the inhibitor slows down progression of osteoarthritis and reduces osteoarthritis-induced pain.

3. The method claimed in claim 1, wherein the inhibitor is selected from the group consisting of a nitrocatechol-derived compound, an antibody or an interfering RNA.

4. The method claimed in claim 1, wherein the inhibitor is a nitrocatechol derivative comprising a catechol part bearing a nitro group in an ortho or para position relative to one of the hydroxyl groups of a catechol part.

5. The method as claimed in claim 4, wherein the nitrocatechol-derived compound is selected from the group consisting of tolcapone, entacapone, opicapone, nitecapone, nebicapone, and mixtures thereof.

6. The method as claimed in claim 1, wherein the inhibitor is designed for intra-articular, oral or subcutaneous administration.

7. The method as claimed in claim 1, wherein the inhibitor is designed for intra-articular administration.

8. The method as claimed in claim 1, wherein the inhibitor is administered in combination with one or more active substances selected from the group consisting of analgesics, non-steroidal anti-inflammatory drugs, steroidal anti-inflammatory drugs, and drugs for osteoarthritis.

9. A method for treating osteoarthritis comprising providing a subject with a pharmaceutical composition comprising one or more COMT inhibitors as claimed in claim 1 and a pharmaceutically acceptable excipient.

10. The method as claimed in claim 9, wherein the composition is designed for intra-articular, oral or subcutaneous administration.

11. The method as claimed in claim 9, wherein the composition further comprises one or more other active substances selected from the group consisting of analgesics, non-steroidal anti-inflammatory drugs, steroidal anti-inflammatory drugs and drugs for osteoarthritis.

12. The method of claim 9, wherein the composition is designed for intra-articular administration.

13. The method of claim 12, wherein the pharmaceutical composition further comprises anti-arthritic agents.

14. The method of claim 9 wherein the pharmaceutical composition is formulated as a sustained-release composition.

15. The method of claim 9 wherein the pharmaceutical composition has for the use as claimed in any one of claims 9 to 13, comprising the one or more COMT inhibitors dispersed in a polymeric fraction comprising a biodegradable and/or biocompatible polymer and mixtures thereof.

16. The method as claimed in claim 3, wherein the nitrocatechol-derived compound is selected from the group consisting of tolcapone, entacapone, opicapone, nitecapone, nebicapone, and mixtures thereof.

17. The method of claim 13 wherein the anti-arthritic agents are selected from the group consisting platelet-rich plasma (PRP), chondroitin, and glucosamine.

18. The method of claim 15 wherein the biocompatible polymer is selected from the group consisting of hyaluronic acid, collagen, chitosan, polylactic acid (PLA), glycolic acid, copolymers of lactic acid and glycolic acid (PLGA), polyoxalates, polycaprolactone, and polyesters.
